(19) United States
(12) Patent Application Publication
(10) Pub. No.: US 2023/0181170 A1
(43) Pub. Date: Jun. 15, 2023

(54) CAPSULE FOR FLUID SAMPLING AND DRUG DELIVERY

(71) Applicant: ANKON TECHNOLOGIES CO., LTD, Wuhan (SG)

(72) Inventors: Tianyi Yangdai, Wuhan (CN); Yuhui BAO, WUHAN (CN); Hangyu PENG, WUHAN (CN); Fanhua MING, Wuhan (CN)

(73) Assignees: ANKON TECHNOLOGIES CO., LTD, Wuhan (CN); ANX IP HOLDING PTE. LTD., Singapore (SG)

(21) Appl. No.: 17/922,560

(22) PCT Filed: Apr. 27, 2021
(86) PCT No.: PCT/CN2021/090279

§ 371 Date: Oct. 31, 2022

(30) Foreign Application Priority Data
Apr. 30, 2020 (CN) 202010360588.7

(51) Int. Cl. A61B 10/00; A61M 31/00

(52) U.S. Cl. CPC A61B 10/0045; A61M 31/002; A61B 2010/0061; A61B 2562/162; A61M 2205/07; A61M 2205/0294

(57) ABSTRACT

The present invention provides a capsule for fluid sampling and drug delivery including: an enclosure; a sampling/drug delivery assembly, including a storage chamber arranged in the enclosure, a first one-way element and a second one-way element communicating with outside of the capsule and the storage chamber, and a pump changing pressure in the storage chamber, wherein the first one-way element and the second one-way element have same direction of access; and a control module, including a microprocessor in communication with the pump. The capsule can realize multiple times of sampling and drug delivery by means of control flow direction of sample or drug through the first one-way element and the second one-way element, and by changing the pressure of the storage chamber through the pump.

### CROSS-REFERENCE OF RELATED APPLICATIONS

The application is a National Phase Application of PCT International Application No. PCT/CN2021/090279, International Filing Date Apr. 27, 2021, published Nov. 4, 2021 as International Publication Number WO2021/218971A1, which claims priority from Chinese Patent Application No. 202010360588.7, filed Apr. 30, 2020, all of which are incorporated herein by reference in their entirety.

### FIELD OF INVENTION

The present invention relates to a medical device, in particular to a capsule capable of sampling fluid and delivering drugs for multiple times.

### BACKGROUND

Site-specific drug delivery and sample collection in gastrointestinal tract are widely used for the study of absorption characteristics of drugs in different positions of the gastrointestinal tract, metabolic status of the gastrointestinal tract and related biochemical characteristics. These studies can lay a solid foundation for research and development of new drugs and provide auxiliary means for detection of gastrointestinal tract diseases.

Currently, there are many system designs for gastrointestinal fluid sampling or drug delivery. However, existing device for gastrointestinal sample collection or drug delivery can only perform a single action. For fluid sampling, if a single sampling fails, for example, air is collected, the device is ineffective and a new device is required.

In addition, existing gastrointestinal fluid sampling device is passive in sampling and its sampling rate and volume of sample that it collects are difficult to estimate. If sampling time is too long, sampling area may change greatly, leading to a low accuracy of positioning. If the sampling time is too short, the collected volume of sample may be insufficient.

To solve the problem, it is necessary to provide an improved capsule for fluid sampling and drug delivery.

### SUMMARY OF THE INVENTION

The present invention provides a capsule capable of sampling fluid and delivering drugs for multiple times.

To achieve the above-mentioned purpose of the present invention, the present invention uses the following technical solutions.

A capsule for fluid sampling and drug delivery comprising: an enclosure; a sampling/drug delivery assembly comprising a storage chamber disposed in the enclosure, a first one-way element and a second one-way element communicating with outside of the capsule and the storage chamber, and a pump changing the pressure in the storage chamber, where the first one-way element and the second one-way element have same direction of access; a control module comprising a microprocessor in communication with the pump.

Further, the pump comprises a deformation membrane communicating with the storage chamber, and a deformation element repeatedly driving at least part of the deformation membrane to change the pressure in the storage chamber;

or, the pump comprises a pneumatic pump that repeatedly pumps air out of and into the storage chamber, and an air storage structure that communicates with the pneumatic pump.

Further, the capsule comprises a spacing wall arranged in the enclosure, and the spacing wall and the enclosure located at a first side of the partition wall constitute the storage chamber. The deformation membrane is disposed on the spacing wall; or the deformation membrane is spaced from the spacing wall, and the deformation element drives the deformation membrane close to or away from the spacing wall.

Further, the deformation element may be at least one of a piezoelectric micropump based on a piezoelectric material, a bimetallic micropump based on a bimetal with dual thermal deformation coefficients, and a micropump based on a shape memory alloy.

Further, the enclosure is provided with a passage communicating with the storage chamber, and the passage comprises a plurality of sample holes disposed on the enclosure and a sample chamber communicating with the plurality of sample holes. The first one-way element connects the sample chamber to the storage chamber.

Further, the enclosure is provided with a second passage communicating with the storage chamber, and the second passage comprises a plurality of sample holes disposed on the enclosure and a sample chamber communicating with the plurality of sample holes. The second one-way element connects the sample chamber to the storage chamber.

Further, an aperture diameter of each of the sample holes is smaller than an aperture diameter of the first one-way element in communication with the sample chamber, and/or the sample chamber has a filter structure.

Further, the capsule comprises a spacing wall arranged in the enclosure, the spacing wall and the enclosure located at the first side of the spacing wall constitute the storage chamber, the first one-way element is arranged adjacent to the spacing wall, and the second one-way element is arranged at an end of the enclosure along an axial direction.

Further, the sampling/drug delivery assembly further comprises an adsorbent material disposed in the storage chamber.

Further, the sampling/drug delivery assembly further comprises a partition wall dividing the storage chamber into a storage chamber body and a sampling passage, a third one-way element arranged on the partition wall to connect the storage chamber body to the sampling passage. The first one-way element connects the outside of the capsule to the sampling passage, the second one-way element connects the storage chamber body to the outside of the capsule, and the pump changes the pressure of the sampling passage.

Further, the capsule comprises a spacing wall arranged in the enclosure, the spacing wall and the enclosure located at the first side of the spacing wall constitute the storage chamber body, the partition wall is parallel to the spacing wall, and the sampling passage is located between the partition wall and the spacing wall.

Further, the sampling/drug delivery assembly further comprises an adsorbent material located within the storage chamber body.

According to all aspects of the present invention, the capsule for fluid sampling and drug delivery can control flow direction of sample or drug through the first one-way element and the second one-way element, and changes the pressure of the storage chamber through power provided by the pump, so that multiple times of sampling and drug delivery can be realized.

### RRIEF DESCRIPTION OF THE DRAWINGS

FIG. 1 is a cross-sectional view of a capsule for fluid sampling and drug delivery cut along an axis direction according to a preferred embodiment of the present invention.

FIG. 2 is a schematic view of the capsule shown in FIG. 1 in an operating state where gastrointestinal fluid flows into a sampling passage of the capsule.

FIG. 3 is a schematic view of the capsule shown in FIG. 1 in an operating state where the gastrointestinal fluid flows from the sampling passage into a storage chamber of the capsule.

FIG. 4 is a cross-sectional view of the capsule for fluid sampling and drug delivery cut along the axis direction according to other preferred embodiment of the present invention.

FIG. 5 is a partial cross-sectional view of the capsule for fluid sampling and drug delivery cut along the axis direction according to other preferred embodiment of the present invention.

FIG. 6 is a partial cross-sectional view of the capsule for fluid sampling and drug delivery cut along the axis direction according to other preferred embodiment of the present invention.

FIG. 7 is a cross-sectional view of the capsule for fluid sampling and drug delivery cut along the axis direction according to a preferred embodiment of the present invention.

FIG. 8 is a schematic view of the capsule shown in FIG. 7 in an operating state where the gastrointestinal fluid flows into the sampling passage.

FIG. 9 is a schematic view of the capsule shown in FIG. 7 in an operating state where the gastrointestinal fluid flows from the sampling passage into the storage chamber.

FIG. 10 is a cross-sectional view of the capsule for fluid sampling and drug delivery cut along the axis direction according to other preferred embodiment of the present invention.

### DETAILED DESCRIPTION

The present invention will be described in detail below with reference to accompanying drawings and preferred embodiments. However, the embodiments are not intended to limit the present invention, and structural, method, or functional changes made by those skilled in the art in accordance with the embodiments are included in the scope of the present invention.

In the figures of the present invention, some dimensions of a structure or portion may be exaggerated relative to other structures or portions for ease of illustration, and thus, are merely used to illustrate basic structure of the present invention.

Referring to FIGS. 1 to 6, showing a preferred embodiment of a capsule 100 for fluid sampling and drug delivery according to the present invention, which comprises an enclosure 1, a sampling/drug delivery assembly 2, and a control module 3. The control module 3 comprises a microprocessor in communication with at least a portion of structures in the sampling/drug delivery assembly 2 to control an operating state of the capsule 100.

The enclosure 1 is biocompatible and cannot be corroded by gastrointestinal fluid, and can be set as transparent or opaque as needed. Moreover, the enclosure 1 is constructed by at least two parts joined together to facilitate arrangement of internal components, and joining position is adaptively designed as required, and is not limited here, and the joining position is not shown in the FIGS.

The a sampling/drug delivery assembly 2 comprises a storage chamber 21 disposed in the enclosure 1, a first one-way element 22 and a second one-way element 23 communicating with outside of the capsule 100 and the storage chamber 21, and a pump 24 changing pressure in the storage chamber 21, wherein the first one-way element 22 and the second one-way element 23 have same direction of access. The pump 26 is an electronically controlled element, communicatively connected to the microprocessor.

It can be understood by those skilled in the art that “the first one-way element 22 and the second one-way element 23 have same direction of access” means that the direction of flow for sampling samples or delivering drugs is the same. The capsule 100 controls the flow direction for sampling samples or delivering drugs through the first one-way element 22 and the second one-way element 23, and changes pressure of the storage chamber 21 by providing power through the pump 24, so that multiple times of fluid sampling and drug delivery can be realized.

When the capsule 100 is used as a sampling capsule for fluid sampling, the storage chamber 21 is empty for storing samples collected at specific sites of gastrointestinal tract. Further, an adsorbent material M having a porous structure is disposed in the storage chamber 21, such as a sponge , etc. During sampling, air is inevitably sucked in. At this point, most of the gastrointestinal fluid, especially thick gastrointestinal fluid, is adsorbed by the adsorbent material M, so that the sucked air and a small volume of the gastrointestinal fluid that may be associated with it can be discharged from the storage chamber 21, and then the ampling is repeated several times by the pump 24. The liquid entering the storage chamber 21 is adsorbed and retained by the adsorbent material M as much as possible. The air is naturally discharged, and after a period of time, the adsorbent material M is almost saturated and sufficient microbial samples can be collected. In addition, the capsule 100 has an active sucking power, making sample collection more rapid and reliable than simple passive collection.

When the capsule 100 is used as a drug delivery capsule for delivery of drugs, the storage chamber 21 is filled with drug to be released, mostly liquid or powder drug. When the capsule 100 reaches a specific site of the gastrointestinal tract, external liquid enters the storage chamber 21 through the cooperation between the pump 24 and the first one-way element 22 and the second one-way element 23, and the drug is then driven out of the capsule 100 to release outward.

Specifically, the capsule 100 comprises a spacing wall 11 arranged in the enclosure 1. The spacing wall 11 and the enclosure 1 located at a first side of the spacing wall 11 constitute the storage chamber 21. In the embodiment shown in FIG. 1, the first one-way element 22 is arranged adjacent to the spacing wall 11, and the second one-way element 23 is arranged at an end of the enclosure 1 along an axial direction.

The first one-way element 22 and the second one-way element 23 are respectively selected from a one-way valve or a one-way membrane. The one-way valve only allows the gastrointestinal fluid and air to pass through in a single direction, and can bear a pressure of 50 kPa ~100 kPa in a reverse direction without infiltration. The one-way valve is installed in such a way that an opening is cut in a corresponding position, and the one-way valve is sealed corresponding to the opening, for example, the one-way valve is sealed and disposed in the opening. Likewise, the one-way membrane only allows fluid and air to pass through in a single direction and is installed in such a way that an opening is cut in a corresponding position, and the one-way membrane covers the opening.

Specifically, the enclosure 1 is provided with a passage communicating with the storage chamber 21, and the first one-way element 22 is arranged on the passage. Specifically, the passage comprises a plurality of sample holes 25 disposed on the enclosure 1 and a sample chamber 26 communicating with the plurality of sample holes 25. The first one-way element 22 connects the sample chamber 26 to the storage chamber 21.

When the first one-way element 22 leads to the storage chamber 21 and the second one-way element 23 leads to outside of the capsule 100, the sample holes 25 are sampling holes 25, which can prevent blocking. During sampling, the gastrointestinal fluid in the gastrointestinal tract enter the sample chamber 26 through the sample hole 25 and converge into the storage chamber 21. Even if some of the sample holes 25 are blocked, rest of them remain in good communicating condition and do not affect sampling. In addition, the gastrointestinal fluid entering through each sample hole 25 can be mixed and buffered through the sample chamber 26 to ensure uniform and smooth sampling.

Further, an aperture diameter of each of the sample holes 25 is smaller than an aperture diameter of the first one-way element 22 communicating with the sample chamber 26, so that substances that can enter the sample chamber 26 through the sample holes 25 do not block the first one-way element 22. Alternatively, the sample chamber 26 comprises a filter structure (not shown in FIGS), such as, but not limited to, a filter screen, to avoid food residues blocking the first one-way element 22. For the filter structure of the sample chamber 26 can be referred to a filter structure designed in Chinese Patent Application No. 201811330328.4. Alternatively, while the aperture diameter of each of the sample holes 25 is smaller than the aperture diameter of the first one-way element 22 communicating with the sample chamber 26, a filter structure may be provided in the sample chamber 26 to achieve a double anti-blocking effect.

The plurality of sample holes 25 are distributed with spacing along lengthwise direction or circumferential direction of the capsule 100 to ensure smooth sampling/drug delivery.

When the second one-way element 23 leads to the storage chamber 21 and the first one-way element 22 leads to the outside of the capsule 100, the gastrointestinal fluid drives the drug from the storage chamber 21 into the sample chamber 26, and then the drug is released to the gastrointestinal tract through a plurality of sample holes 25, which expands the range of drug delivery.

Alternatively, the enclosure 1 is provided with a second passage (not shown in FIGS), the second one-way element 23 can also connect the storage chamber 21 to the outside of the capsule 100 through the second passage. The second passage has the same structure as the passage, and the second passage specifically comprises a plurality of sample holes opened on the enclosure, and a sample chamber connected to the plurality of sample holes; the second one-way element connects the sample chamber to the storage chamber.

Preferably, both the first one-way element 22 and the second one-way element 23 connects the storage chamber 21 to the outside of the capsule 100 through the above passages comprising sample holes 25 and the sample chamber 26. Regardless of the direction of access of the first one-way element 22 and the second one-way element 23, the blocking prevention effect can be achieved at the time of sampling, and the wide range of drug delivery can be achieved at the time of drug delivery.

The pump 24 is used to change the pressure in the storage chamber 21 and cooperate with the direction of access of the first one-way element 22, a third one-way element 28 and the second one-way element 23 to complete sampling or drug delivery.

In one embodiment, as shown in FIGS. 1 to 3, and 5, the first one-way element 22 leads to the storage chamber 21, the second one-way element 23 leads to the outside of the capsule 100, and the gastrointestinal fluid flows into the storage chamber 21 through the first one-way element 22 and can be discharged to the outside of the capsule 100 through the second one-way element 23.

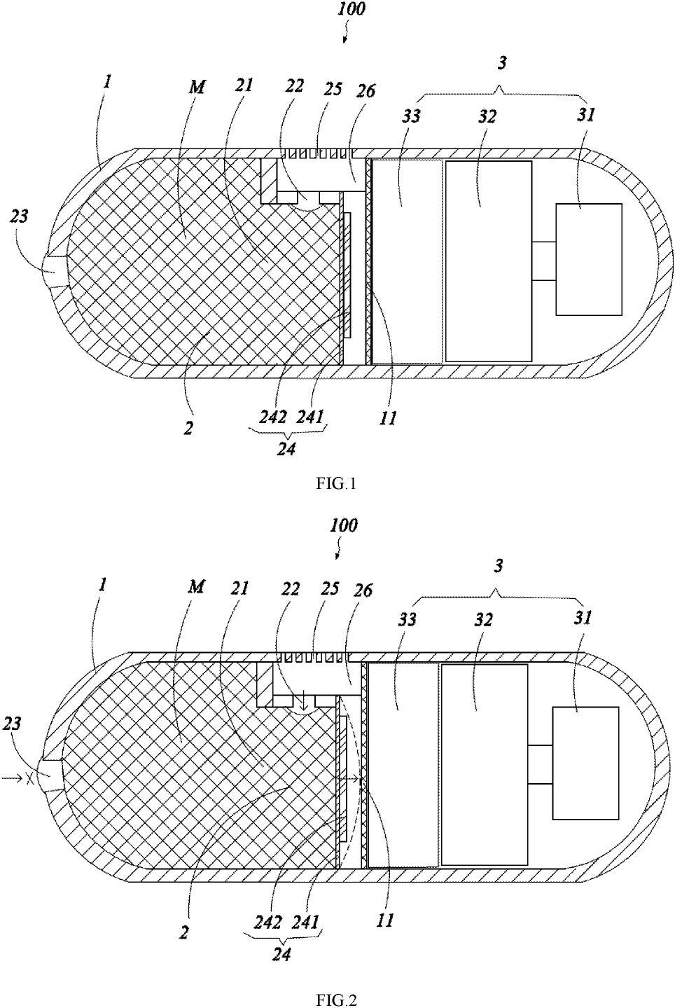

The specific sampling process is: as shown in FIG. 2, the pump 24 reduces the pressure of the storage chamber 21, and the gastrointestinal fluid enters the storage chamber 21 through the first one-way element 22 under the action of internal and external pressure difference to complete the sampling. If air enters the storage chamber 21 during sampling, the sampling process is repeated, wherein sample is re-collected and the air is discharged from the capsule through the second one-way element 23, and the sampling is completed after multiple times of sampling.

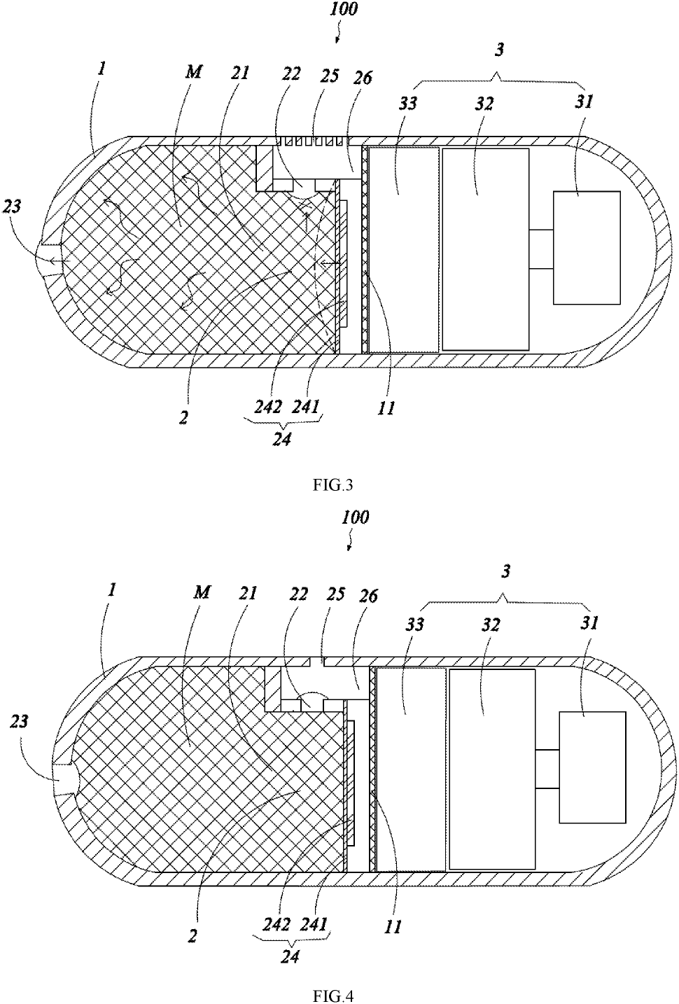

The specific drug delivery process is: Referring to FIG. 2, the pump 24 reduces the pressure in the storage chamber 21, and the gastrointestinal fluid enters the storage chamber 21 through the first one-way element 22 under the action of the internal and external pressure difference. Then, as shown in FIG. 3, the pump 24 increases the pressure in the storage chamber 21, and the gastrointestinal fluid drives the drugs stored in the storage chamber 21 to flow outward from the second one-way element 23 into the gastrointestinal tract.

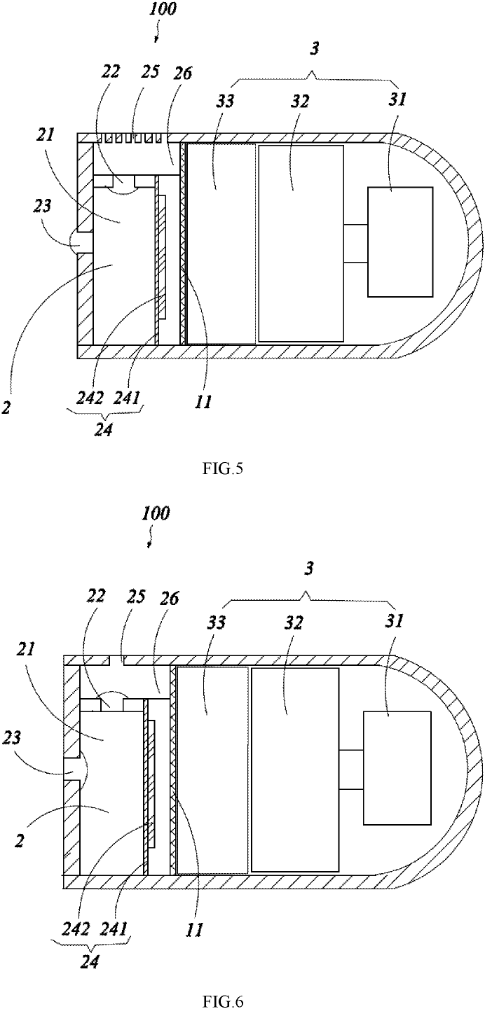

In another embodiment, as shown in FIGS. 4 and 6, the second one-way element 23 leads to the storage chamber 21, the first one-way element 22 leads to the outside of the delivery capsule 100, and the gastrointestinal fluid flows into the storage chamber 21 through the second one-way element 23 and can flow out of the capsule 100 through the first one-way element 22.

The specific sampling process is: the pump 24 reduces the pressure of the storage chamber 21, and the gastrointestinal fluid enters the storage chamber 21 through the second one-way element 23 to complete the sampling. If air enters the storage chamber 21 during sampling, the sampling process is repeated, where sample is re-collected and the air is discharged from the capsule 100 through the second one-way element 22, and the sampling is completed after multiple times of sampling.

The specific drug delivery process is: the pump 24 reduces the pressure in the storage chamber 21, and the gastrointestinal fluid enters the storage chamber 21 through the second one-way element 23 under the action of the internal and external pressure difference. Then, the pump 24 increases the pressure in the storage chamber 21, and the gastrointestinal fluid pushes the drug to be discharged from the capsule through the first one-way element 22.

In one embodiment, the pump 24 comprises a deformation membrane 241 communicating with the storage chamber 21, and a deformation element 242 repeatedly driving at least part of the deformation membrane 241 to change the pressure in the storage chamber 21.

Specifically, the deformation membrane 241 is disposed on the spacing wall 11. When the deformation membrane 241 is deformed, a volume of the storage chamber 21 changes, and thus the pressure in the storage chamber 21 changes. Alternatively, the deformation membrane 241 is spaced from the spacing wall 11, and the deformation membrane 241 can be located in or outside the storage chamber 21. At this point, it can also be understood that a pump cavity is formed between the deformation membrane 241 and the spacing wall 11, and the deformation element 242 drives the deformation membrane 241 to be close to or away from the spacing wall 11. When the deformation membrane 241 is deformed, the volume of the storage chamber 21 changes, and thus the pressure in the storage chamber 21 changes.

The deformation element 242 is a piezoelectric micropump based on a piezoelectric material. When a voltage is applied to the piezoelectric micropump, the piezoelectric material can bend, driving the deformation membrane 241 to bend to increase or decrease the pressure in the storage chamber 21.

Take sampling as an example. Referring to FIG. 2 and FIG. 3, when a voltage is applied to the piezoelectric micropump, the piezoelectric material bends away from the storage chamber 21, driving the deformation membrane 241 to bend away from the storage chamber 21 to increase the volume of the storage chamber 21 and reduce the pressure of the storage chamber 21, and the gastrointestinal fluid flows into the storage chamber 21 through the first one-way element 22 under the action of the internal and external pressure difference. When the applied voltage is eliminated, the piezoelectric material is restored to its original shape, driving the deformation membrane 241 to restore to its original shape to complete sampling. If air is collected, a reverse voltage can be applied so that the piezoelectric material bends toward the storage chamber 21, and the pressure in the storage chamber 21 increases so that the collected air or part of the gastrointestinal fluid is discharged out of the capsule 100 through the second one-way element 23, and then the above operation is repeated to complete the sampling. In this process, the liquid is viscous and the fluidity is lower than that of air, so the air is preferentially discharged. However, in embodiments with the adsorbent material M, the collected liquid sample is substantially retained within the storage chamber 21.

Take drug delivery as an example. When a voltage is applied to the piezoelectric micropump, and the piezoelectric material bends away from the storage chamber 21, driving the deformation membrane 241 to bend away from the storage chamber 21 to increase the volume of the storage chamber 21 and reduce the pressure of the storage chamber 21. The gastrointestinal fluid enters the storage chamber 21 through the first one-way element 22 under the action of the internal and external pressure difference. When the applied voltage is removed, the piezoelectric material is restored to its original shape, driving the deformation membrane 241 to restore to its original shape, or when a reverse voltage is applied, the piezoelectric material bends toward the storage chamber 21, and the pressure in the storage chamber 21 increases, so that the gastrointestinal fluid drives the drug in the storage chamber 21 to flow outward from the second one-way element 23 into the gastrointestinal tract.

As described above, when a voltage is periodically applied to the piezoelectric micropump, the piezoelectric micropump periodically deforms and recovers the deformation membrane 241 to generate vibration, so as to perform multiple sampling or drug delivery to avoid various disadvantages of one sampling and drug delivery.

Alternatively, the deformation element 242 is a bimetallic micropump based on a bimetal with dual deformation coefficients, and the bimetallic micropump is composed of two different metal diaphragms that are relatively fixed. Since the deformation coefficients of the two metal diaphragms are different, when they are heated by a heating element, such as a resistor, the two metal diaphragms are deformed to different degrees, making the bimetallic micropump deformed, and thus deforming the deformation membrane 241, and then changing the pressure of the storage chamber 21.

As shown in FIG. 2, during heating, the bimetallic micropump bends away from the storage chamber 21, thereby causing the deformation membrane 241 to bend away from the storage chamber 21, reducing the pressure of the storage chamber 21. After the heating is stopped and the temperature is restored, the shape of the bimetallic micropump is restored, the shape of the deformation membrane 241 is restored, and the pressure in the storage chamber 21 is increased. In this embodiment, the principle and process of sampling and drug delivery based on pressure change are the same as those in the above embodiments, and will not be repeated here.

Alternatively, the deformation element 242 is a micropump based on a shape memory alloy. The micropump based on the shape memory alloy is made of the shape memory alloy. When the temperature of the shape memory alloy is raised to a certain value by using a heating element, the shape memory alloy can deform and cause the deformation membrane 241 to deform, thereby changing the pressure of the storage chamber 21.

As shown in FIG. 2, during heating, the micropump based on the shape memory alloy bends away from the storage chamber 21, thereby causing the deformation membrane 241 to bend away from the storage chamber 21 and reducing the pressure of the storage chamber 21. After the heating is stopped and the temperature is restored, the shape of the micropump based on the shape memory alloy is restored, the shape of the deformation membrane 241 is restored, and the pressure in the storage chamber 21 is increased. In this embodiment, the principle and process of sampling and drug delivery based on pressure changing are the same as those in the above embodiments, and will not be repeated here.

In another embodiment, the pump 24 comprises a pneumatic pump that can repeatedly pump air out of and into the storage chamber 21, and an air storage structure that communicates with the pneumatic pump. The pneumatic pump changes air pressure of the storage chamber 21 by pumping the air in the storage chamber 21 into the air storage structure or pumping the air in the air storage structure into the storage chamber 21, thereby controlling the flow of the sample or drug. The volume of the air storage structure is set such that when the air flows in the storage chamber 21 and the air storage structure, a sufficient pressure difference can be generated to meet the requirements of sampling or drug delivery.

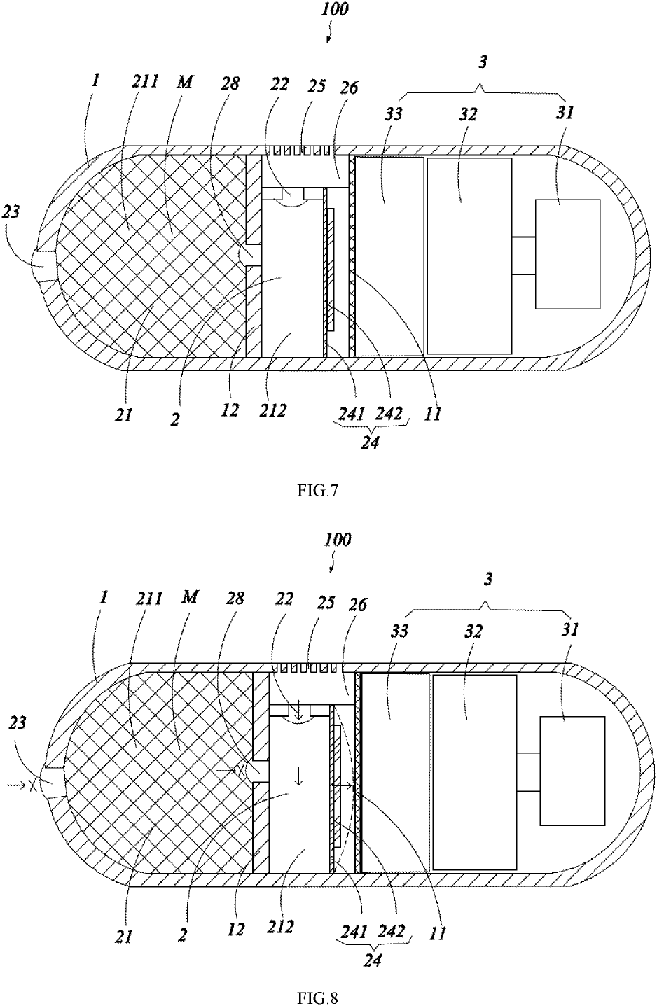

Referring to FIGS. 7 to 10, based on any of the above embodiments, the sampling/drug delivery assembly 2 further comprises a partition wall 12 dividing the storage chamber 21 into a storage chamber body 211 and a sampling passage 212, a third one-way 28 element arranged on the partition wall 12 to connect the storage chamber body 211 to the sampling passage 212. The first one-way element 22 connects the sampling passage 212 to the outside of the capsule 100, the second one-way element 23 connects the storage chamber body 211 to the outside of the capsule 100, and the pump 24 changes the pressure of the sampling passage 212.

The partition wall 12 defines a smaller sampling passage 212 within the storage chamber 21 for temporarily storing the sample/drug during use, the pump 24 changes the pressure of the sampling passage 212 and can reduce the pressure requirement of the pump 24. Based on the pump 24 of same suction capacity, the internal pressure of the sampling passage 212 varies greatly, increasing the power of sampling and drug delivery.

Specifically, the partition wall 12 is parallel to the spacing wall 11, the sampling passage 212 is located between the partition wall 12 and the spacing wall 11, and the pump 24 is arranged on the side of the partition wall 12 facing the spacing wall 11. The specific structure and arrangement mode are as described above, and will not be repeated here.

In one embodiment, the third one-way element 28 is located at the center of the partition wall 12 so that the sample/drug can pass through it smoothly.

Preferably, the sampling/drug delivery assembly 2 further comprises an adsorption material M located in the storage chamber body 211 for adsorbing the gastrointestinal fluid during sampling.

The operating principle of the capsule 100 having the sampling passage 212 can be described below.

In one embodiment, as shown in FIGS. 7 to 9, the first one-way element 22 leads to the sampling passage 212, the third one-way element 28 leads to the storage chamber body 211, and the second one-way element 23 leads to the outside of the capsule 100.

The specific sampling process is: as shown in FIG. 8, the pump 24 reduces the pressure of the sampling passage 212, and the gastrointestinal fluid enters the sampling passage 212 through the first one-way element 22 under the action of the internal and external pressure difference. Then, as shown in FIG. 9, the pump 24 increases the pressure in the sampling passage 212, and the gastrointestinal fluid enters the storage chamber body 211 through the third one-way element 28 to complete sampling. If air enters the storage chamber body 211 during sampling, the sampling process is repeated, where sample is re-collected and the air is discharged from the capsule 100 through the second one-way element 23, and the sampling is completed after multiple times of sampling. Preferably, the adsorption material M with a porous structure in the storage chamber body 211 can collect a sufficient volume of microbial samples after multiple times of sampling.

The specific drug delivery process is: referring to FIG. 8, the pump 24 reduces the pressure of the sampling passage 212, and the gastrointestinal fluid enters the sampling passage 212 through the first one-way element 22 under the action of the internal and external pressure difference. Then, referring to FIG. 9, the pump 24 increases the pressure in the sampling passage 212, and the gastrointestinal fluid enters the storage chamber body 211 through the third one-way element 28, and drives the drug placed in the storage chamber 211 to flow outward from the second one-way element 23 into the gastrointestinal tract to complete the drug delivery.

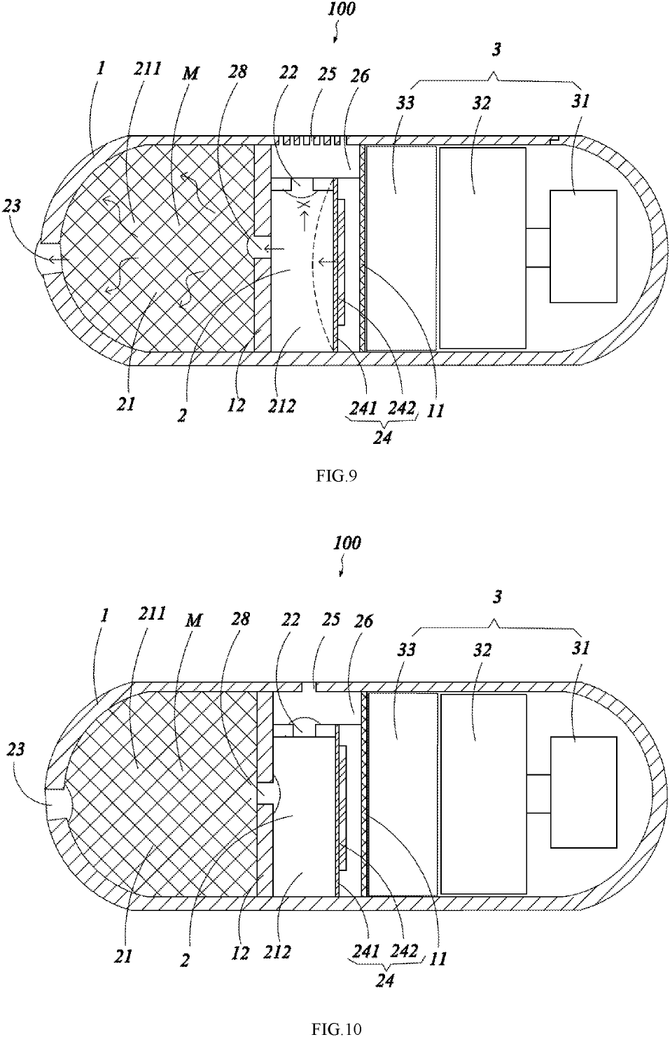

In another embodiment, as shown in FIG. 10, the second one-way element 23 leads to the storage chamber body 211, the third one-way element 28 leads to the sampling passage 212, and the first one-way element 22 leads to the outside of the capsule 100.

The specific sampling process is: the pump 24 reduces the pressure of the sampling passage 212, the air in the storage chamber body 211 enters the sampling passage 212 through the third one-way element 28 to reduce the pressure of the storage chamber body 211, and the gastrointestinal fluid enters the storage chamber body 211 through the second one-way element 23 under the action of the internal and external pressure difference, to complete sampling. Since the volume of the storage chamber body 211 is large, when it cannot be filled in one sampling, the above operation can be repeated for multiple sampling. In addition, if air enters the storage chamber body 211 during the sampling, the above sampling process is repeated, and the air is re-sampled and discharged from the capsule 100 through the third one-way element 28, the sampling passage 212, and the first one-way element 22, and the sampling is re-completed after multiple times of sampling. Preferably, the adsorption material M with a porous structure in the storage chamber body 211 can collect a sufficient volume of microbial samples after multiple times of sampling.

The specific drug delivery process is: the pump 24 reduces the pressure of the sampling passage 212, the air/drug in the storage chamber body 211 enters the sampling passage 212 through the third one-way element 28 to reduce the pressure of the storage chamber body 211, and the gastrointestinal fluid enters the storage chamber body 211 through the second one-way element 23 under the action of the internal and external pressure difference; the pump 24 increases the pressure of the sampling passage 212, the air/drug previously entering the sampling passage 212 is discharged from the capsule 100 through the first one-way element 22. The pressure of the sampling passage 212 is reduced and increased by the pump 24 many times, so that the gastrointestinal fluid entering the storage chamber body 211 pushes the drug through the third one-way element 28 into the sampling passage 212, and then the drug is discharged from the capsule 100 through the first one-way element 22 to realize multiple times of drug delivery.

In general, sampling or drug delivery may be performed when the capsule 100 is at a site of the gastrointestinal tract to be examined, or reaches a site of the gastrointestinal tract with lesions.

The control module 3 further comprises a sensor 31 for collecting physiological parameters and/or image information in the gastrointestinal tract, and the sensor 31 communicates with the microprocessor. The sensor 31 is at least an image sensor, or a pH sensor, or an ultrasonic sensor. When the sensor 31 comprises an image sensor, part of the enclosure 1 is transparent, and when the sensor 31 comprises a pH sensor, the enclosure 1 has a window. The specific method of determining which site of the gastrointestinal tract the capsule 100 is in, based on images and pH values obtained by the sensor 31, can be any method in the prior art, and will not be repeated herein.

In addition to the sensor 31, the control module 3 can further comprise a storage module for storing physiological parameters and image information in different regions of the gastrointestinal tract, where the storage module communicates with the microprocessor. The parameters and image information comprise normal physiological parameters or normal image information, and physiological parameters or image information in case of possible lesions. When the sensor 31 collects the physiological parameters and/or image information in the gastrointestinal tract, the microprocessor compares the above information with the information in the storage module to determine whether the capsule 100 reaches a position at which the sample is to be collected or drug is to be released.

Alternatively, in addition to the sensor 31, the control module 3 further comprises a wireless transmission module for communicating with an external processing terminal. When the sensor 31 collects the physiological parameters and/or image information in the gastrointestinal tract, the information is transmitted to the external processing terminal, and the external processing terminal analyzes the information and determines whether the capsule reaches 100 has reached the position at which the sample is to be collected or drug is to be released.

In addition, the control module 3 further comprises a battery 32 for supplying power to other components of the capsule 100, and the microprocessor and the wireless transmission module are generally integrated on a same circuit board 33.

The capsule for fluid sampling and drug delivery of the present invention can communicate with the external processing terminal through the control module 3, and the control module 3 communicates with the external processing terminal by any of the methods in the prior art, and it will not be repeated herein.

To sum up, the capsule 100 for fluid sampling and drug delivery controls the flow direction of sample or drug through the first one-way element 22 and the second one-way element 23, and changes the pressure of the storage chamber 21 through the power provided by the pump 24, so that multiple times of sampling and drug delivery can be realized.

It should be understood that, although the specification is described in terms of embodiments, not every embodiment merely comprises an independent technical solution, and the specification is described in this manner only for clarity. Those skilled in the art should have the specification as a whole, and the technical solutions in each embodiment may also be combined as appropriate to form other embodiments that can be understood by those skilled in the art.

The series of detailed descriptions set forth above are only specific descriptions of feasible embodiments of the present invention and are not intended to limit the scope of protection of the present invention. Any equivalent embodiments or modifications made without departing from the spirit of the art of the present invention shall be included within the scope of protection of the present invention.

1. A capsule for fluid sampling and drug delivery, comprising:
an enclosure;
a sampling/drug delivery assembly comprising a storage chamber disposed in the enclosure, a first one-way element and a second one-way element communicating with outside of the capsule and the storage chamber, and a pump changing pressure in the storage chamber, wherein the first one-way element and the second one-way element have same direction of access; and
a control module comprising a microprocessor in communication with the pump;
wherein the capsule comprises a spacing wall arranged in the enclosure, and the spacing wall and the enclosure located at a first side of the partition wall constitute the storage chamber;
wherein the enclosure is provided with a passage communicating with the storage chamber, and the passage comprises a plurality of sample holes disposed on the enclosure and a sample chamber communicating with the plurality of sample holes; and wherein the first one-way element connects the sample chamber to the storage chamber.

2. The capsule of claim 1, wherein the pump comprises a deformation membrane communicating with the storage chamber, and a deformation element repeatedly driving at least part of the deformation membrane to change the pressure in the storage chamber;
or, the pump comprises a pneumatic pump that repeatedly pumps air out of and into the storage chamber, and an air storage structure that communicates with the pneumatic pump.

3. The capsule of claim 2, wherein the deformation membrane is disposed on the spacing wall; or the deformation membrane is spaced from the spacing wall, and the deformation element drives the deformation membrane close to or away from the spacing wall.

4. The capsule of claim 2, wherein the deformation element is at least one of a piezoelectric micropump based on a piezoelectric material, a bimetallic micropump based on a bimetal with dual thermal deformation coefficients, and a micropump based on a shape memory alloy.

5. (canceled)

6. The capsule of claim 1, wherein the enclosure is provided with a second passage communicating with the storage chamber, and the second passage comprises a plurality of sample holes disposed on the enclosure and a sample chamber communicating with the plurality of sample holes; and wherein the second one-way element connects the sample chamber to the storage chamber.

7. The capsule of claim 1, wherein an aperture diameter of each of the sample holes is smaller than an aperture diameter of the first one-way element in communication with the sample chamber, and/or the sample chamber comprises a filter structure.

8. The capsule of claim 1, wherein the spacing wall, and the second one-way element is arranged at an end of the enclosure along an axial direction.

9. The capsule of claim 1, wherein the sampling/drug delivery assembly further comprises an adsorbent material disposed in the storage chamber.

10. The capsule of claim 1, wherein the sampling/drug delivery assembly further comprises a partition wall dividing the storage chamber into a storage chamber body and a sampling passage, a third one-way element arranged on the partition wall to connect the storage chamber body to the sampling passage; and wherein the first one-way element connects the outside of the capsule to the sampling passage, the second one-way element connects the storage chamber body to the outside of the capsule, and the pump changes the pressure of the sampling passage.

11. The capsule of claim 10, wherein the capsule comprises a spacing wall disposed in the enclosure, the spacing wall and the enclosure located at the first side of the spacing wall constitute the storage chamber body, the partition wall is parallel to the spacing wall, and the sampling passage is located between the partition wall and the spacing wall.
